(19) United States
(12) Patent Application Publication
(10) Pub. No.: US 2025/0266135 A1
(43) Pub. Date: Aug. 21, 2025

(54) DIGITAL PATHOLOGY OF BREAST CANCER BASED ON A SINGLE CELL MASS SPECTROMETRY IMAGING DATABASE

(71) Applicants: UNIVERSITEIT MAASTRICHT, MAASTRICHT (NL); ACADEMISCH ZIEKENHUIS MAASTRICHT, MAASTRICHT (NL)

(72) Inventors: Ronald Martinus Alexander HEEREN, MAASTRICHT (NL); Eva CUYPERS, MAASTRICHT (NL)

(21) Appl. No.: 18/562,037

(22) PCT Filed: May 18, 2022
(86) PCT No.: PCT/EP2022/063435

§ 371 Date: Nov. 17, 2023

(30) Foreign Application Priority Data
May 19, 2021 (EP) 21174636.7

(51) Int. Cl. G16H 10/40; G06T 7/00; G06V 20/69

(52) U.S. Cl. CPC G16H 10/40; G06T 7/0014; G06V 20/698; G06T 2207/10072; G06T 2207/30024; G06T 2207/30068

(57) ABSTRACT

The invention relates to methods using an automated system for characterizing or analyzing single cells in a sample based on mass spectrometry imaging. The methods find use in among other in pathology and diagnosis. The invention further relates to a method for constructing a reference database for use herein.

### CROSS REFERENCE TO RELATED APPLICATIONS

This application is a national stage application under 35 U.S.C. § 371 of PCT/EP2022/063435 filed May 18, 2022, which claims priority to EP Application Serial No. 21174636.7 filed May 19, 2021, each of which are hereby incorporated by reference in their entirety.

### FIELD OF THE INVENTION

The invention relates to the field of digital pathology. More specifically, the method relates to an automated system to analyze or characterize single cells in a sample based on molecular profiles generated by mass spectrometry imaging.

### BACKGROUND OF THE INVENTION

Digital pathology of cancer, infection or metabolic diseases is challenging due to its complex heterogeneity of cellular subtypes. The ability to directly identify and visualize these subtype distribution at the single cell level within a tissue section would bring digital pathology to the next level and opens up fast diagnosis and prognosis. In modern clinical practice, digital pathology offers many advantages over microscopic examination of glass slides alone. Digital images have demonstrated to improve the overall analysis, reduce the number of errors, and provide better contextual views of a tissue under study. Advances in machine learning have enabled the synergy of artificial intelligence and digital pathology, which in theory offers image-based diagnosis possibilities. All of these innovative approaches depend on information-rich images, rich in spatial detail, spectral detail, or molecular detail. In practice, the genomic and transcriptomic heterogeneity of e.g. cancer makes digitalisation of the pathological workflow very complicated and challenging. Genomic and transcriptomic alterations and tumor microenvironment research of cancer and their important implications in prediction of therapy response and survival rate are described extensively in literature.

For example in breast cancer, oestrogen receptor (ER) and progesterone receptor (PR) expression are generally considered as prognostic as well as diagnostic markers and as predictors of hormone therapy response. They also provide some information on response to chemotherapy: ER-tumors respond better than ER+ tumors. Human epidermal growth factor receptor 2 (HER2) overexpression and/or gene amplification predict response to anti-HER2 targeted therapy. It also provides prognostic information and can be used to help in diagnosis (i.e., Paget's disease).

Single cell Mass Spectrometry Imaging (MSI) has been described for example in Zavalin et al., J Mass Spectrom. 2012 November; 47 (11): i. doi: 10.1002/jms.3132. The described method requires a special transmission geometry setup.

Single cell MSI based classification has been described in Šcupáková et al., Ang. Chemie Int. Ed. (2020) 59 17447-17450. The journal article does not describe a recognition system based on single cell molecular information and database, but employs morphometry to classify cells without linking them to diagnostic profile.

U.S. Pat. No. 7,228,239 B1 describes a method for classifying mass spectra to discriminate the absence or existence of a condition. The mass spectra may include raw mass spectrum intensity signals or may include intensity signals that have been pre-processed. The method and systems include determining a first or higher order derivative of the signals of the mass spectra, or any linear combination of the signal and a derivative of the signal, to form a mass spectra data set for training a classifier. The mass spectra data set is provided as input to train a classifier, such as a linear discrimination classifier. The classifier trained with the derivative-based mass spectra data set then classifies mass spectra samples to improve discriminating between the absence or existence of a condition. Although some degree of automation is reached by this method, the method is not based on single cell mass spectrometry, and moreover does not describe the use of mass spectrometry imaging, therefore information on the single cell level and localization of specific cells within the sample is lost, diminishing its use as an on the fly analysis or diagnostic tool.

Therefore there is a need for improved automated imaging based classification methods based on single cell mass spectrometry. The afore mentioned drawbacks of the current state of digital pathology are overcome by the invention as defined in the appended claims.

Today, immunohistochemistry is standardly used to test these protein expressions although there are some major disadvantages of the technique: time consuming, stains are not standardised worldwide and it is subjective to human error, interpretation (not yet amendable to automated interpretation) and pre-analytical variability (fixation time and method) with lacking robust internal controls. Assessment of ER-status based on RNA expression can provide more objective, quantitative and reproducible test results. However this is challenging to apply to fixed tissue and it is very time consuming.

### SUMMARY OF THE INVENTION

The present invention relates to a method for identifying one or more single cells in a sample, the method comprising: performing mass spectrometry imaging (MSI) on the one or more single cells in the sample to obtain single cell mass spectrometry imaging data from the one or more single cells in the sample; applying to the obtained single cell mass spectrometry imaging data from the one or more single cells in the sample a recognition model to identify the one or more single cells in the sample; wherein the recognition model comprises a reference database to which a dimensionality reduction algorithm has been applied, wherein the dimensionality reduction algorithm has classified the data from the reference database in classes representing distinct cell types, obtaining classified reference data, wherein the applying of the recognition model to the obtained single cell mass spectrometry imaging data from the one or more single cells in the sample comprises assigning a probability that the obtained single cell mass spectrometry imaging data from the one or more single cells in the sample belong to a class within the classified reference database, and wherein the one or more single cells in the sample is identified based on the highest probability; wherein the reference database comprises single cell mass spectrometry imaging data, obtained by mass spectrometry imaging, from at least two distinct cell types with known and differing characteristics, wherein the single cell mass spectrometry imaging data in the reference database and the single cell mass spectrometry imaging data obtained from the sample comprise for each cell at least one spectra.

The invention further relates to a method of diagnosing a subject with cancer using the method according to the invention, wherein the sample is a tumor sample obtained from the subject, and wherein the diagnosis is based on the characteristic or characteristics from the reference cell which has or have been assigned to the cell in the sample, and wherein the two or more distinct cell types in the reference library are two or more distinct tumor cells, preferably two or more cancer cell lines.

The invention further relates to a method for constructing a recognition model for identifying one or more single cells in a sample, the method comprising the steps of: providing at least two samples each comprising distinct cell types; performing single cell mass spectrometry imaging on at least two single cells in each sample in a predefined m/z range, wherein for each single cell a minimum of one, preferably three, single cell mass spectrometry imaging datasets are obtained; building a database comprising for each cell line the single cell mass spectrometry imaging data within the predefined m/z range and the corresponding subcellular localization data; and constructing the recognition model by applying a dimensionality reduction algorithm to the reference database, preferably wherein the dimensionality reduction algorithm is selected from Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbor Embedding (t-SNE), preferably wherein the at least two samples comprising distinct cell types are at least two distinct cell lines.

### DETAILED DESCRIPTION OF THE INVENTION

The inventors here describe a method for identifying cells in a sample based on MSI technology and machine learning. The method allows “on the fly” identification or characterization of cells on a microscope slide, and finds use in e.g. diagnosis or sample analysis applications. As a proof of concept the inventors provide on the fly diagnosis of a breast cancer sample based on a trained dataset.

Here, with matrix-assisted laser desorption/ionization mass spectrometry imaging (MALDI-MSI), with subcellular resolution the molecular profile distributions of 14 different breast cancer types were visualized in a mass range of 200-1200 m/z representing different molecular expressions. The inventors show that these in vitro cultured and acquired cellular mass profiles are representative for in tissue cell subtypes and that our recognition models can identify the breast cancer subtype on a single cell level in xenografts. As an ultimate proof of concept, the inventors performed ‘on-the-fly’ cell typing on MDA-MB-231 and MDA-MB-468 xenografts.

In order to be able to use these recognition models as standard diagnostic and prognostic tools, there are two requirements that need to be fulfilled: 1) cell profiles are preferably MALDI-instrument independent so cell type visualization can be carried out based on our presented models without the need of separate models for every MALDI-instrument type; 2) cell profiles need to have in-tissue relevance in order to be able to recognize the correct cell type in a clinical relevant environment, namely resected patient tissue samples.

The method of the invention is not limited to specific types of molecules, and can thus be based on proteins, peptides, metabolites, polynucleotides and lipids. Although the method does not restrict to a specific type of molecule, it was founds that variation in lipids contributed greatly to the model. Little is known if transcriptomic differences are reflected in the local lipidome of breast cancer subtypes. Insights in the function and mechanism of lipid molecules and their role in the diagnosis and prognosis of breast cancer is steadily increasing. It has been shown that the heterogeneity of breast cancer subtypes is reflected in the expression levels of enzymes in lipid metabolism and as a consequence of lipid levels and ratios. Moreover, since breast cancer research has identified tumor subtypes are associated with distinct clinical behaviors, in-depth unravelling the molecular differences of these subtypes and developing an online subtype recognition method is of great prognostic and therapeutic medical value.

In order to build single cell, molecular models, there are two main challenges: 1) subcellular level spatial resolution is required with a minimum of 3 pixels per cell, meaning that for cells of 20 μm diameter, a pixel size of 5 μm is needed, and 2) this high spatial resolution should be combined with a high sensitivity mode in order to acquire as much molecular information as possible in a broad m/z range. At the moment, these two requirements can be found in timsTOF flex MALDI-2 technology which increases the sensitivity by about 3 orders of magnitude. The acquired timsTOF flex MALDI-2 single cell profiles (and accompanying recognition models) were found to be comparable to spectra acquired with other MALDI-MSI techniques (Time-Of-Flight and orbitrap).

Therefore, in a first aspect the invention relates to a method for identifying one or more single cells in a sample, the method comprising: performing mass spectrometry imaging (MSI) on the cell in the sample to obtain single cell mass spectrometry imaging data from the cell in the sample; applying to the obtained single cell mass spectrometry imaging data from the cell in the sample a recognition model to identify the cell in the sample; wherein the recognition model comprises a reference database to which a dimensionality reduction algorithm has been applied, wherein the dimensionality reduction algorithm has classified the data from the reference database in classes representing distinct cell types, obtaining classified reference data, wherein the applying of the recognition model to the obtained single cell mass spectrometry imaging data from the cell in the sample comprises assigning a probability that the obtained single cell mass spectrometry imaging data from the cell in the sample belongs to a class within the classified reference database, and wherein the cell in the sample is identified based on the highest probability; wherein the reference database comprises single cell mass spectrometry imaging data, obtained by mass spectrometry imaging, from at least two distinct cell types with known and differing characteristics, wherein the single cell mass spectrometry imaging data in the reference database and the single cell mass spectrometry imaging data obtained from the sample comprise for each cell at least one spectra.

The inventors have conducted for the first time a molecular profile analysis of cancer cells at subcellular spatial resolution applying mass spectrometry imaging to investigate subtype heterogeneity and the use hereof in digital pathology. A robust and repeatable method has been developed to grow cells on conductive glass slides that can directly be used to acquire mass spectrometry imaging data at subcellular spatial resolution (FIG. 2). Moreover, it is shown for the first time that this method provides a sensitivity that is high enough to perform DDA analysis in order to confidently identify single cell MS2 lipid profiles. This method makes it possible to investigate identified compound distributions within a single cell enabling a greater understanding of cellular processes and alterations. Applying this method and developing single cell databases of other cancer related cell types significantly enhances our knowledge and insight into compound distributions, cell-cell interactions and cell specific therapy response.

The cellular chemical profile was acquired between 200-1200 m/z, nevertheless, subtypes appear to have the best separation rate in PCA/LDA analysis in the lipid mass range of 600-950 m/z. Investigating the heat maps (FIG. 3) and FIG. 10 in more detail, it appears that most lipids are present in all cell types, but lipid ratios are responsible for subtype heterogeneity. It needs to be noted that, within a single cell, these lipids have specific intracellular distributions, as shown in FIG. 1 and by the error bars in FIG. 10. The possibility to visualize these lipid distributions at a sub-cellular level opens up possibilities for intracellular pathway studies in the future. It is important to mention that for our cell type recognition model, the mean spectrum of the full cell ROI was used and thus intracellular distributions were not taken into consideration. Moreover, we want to emphasize that our model is generated based on the full mass spectrum meaning that also other compound classes, including potential molecular markers, may contribute to the excellent cross-validation results of our PCA/LDA recognition models. Therefore, the presented models are invaluable in pathway and molecular markers research as they can point out significant differences in molecular profiles.

Comparative analysis of mass profiles obtained on different mass spectrometry imaging instruments (with similar MALDI sources) revealed no significant differences between instruments when comparing the main differentiating lipid ratios. Mass spectra showed great similarities and, as shown in FIG. 5 and by our statistical analysis, differentiating lipids show the same intensity ratios for all instruments evaluated. It is emphasized that since the recognition model is based on ratios rather than absolute abundancies, it is important to focus on these ratios when comparing the different MALDI instruments. The inventors were able to prove successful application of the timsTOF flex MALDI-2 model on other MALDI-TOF equipment including Synapt G2-Si HDMS (FIG. 6 and supplemental video). As a consequence, the constructed model can be considered as instrument independent.

It is known that in breast cancer ER/PR/HER2 status profiles provides direct insight into tumor aggressiveness and the overall patient survival rate. Moreover, these status profiles are described to have a predictive value regarding response to chemotherapy and drug treatment. Therefore, any model that can rapidly determine the ER, PR, HER2 status of breast cancer tissue has substantial predictive and prognostic potential. Here, the possibility to rapidly detect the receptor and HER2 status is created without the use of expensive labels or antibodies. Moreover, it is much less susceptible to technical variance (e.g. fixation time in immunohistochemistry) and subjective scoring and interpretation. Therefore, the developed method can be considered as a great step forward in fast and objective diagnostics that directly leads to better patient treatment.

It also opens great opportunities for studying the effect of new treatment possibilities on tumor heterogeneity and growth, possibly leading to future personalized medicine.

Within the last decade, the main focus in mass spectrometry imaging was set on technical advances with great improvement in speed and spatial resolution. However, vendor-independent data analysis software, including model builders and online recognition software, is still lacking. This research, for the first time showing the direct implementation of mass spectrometry imaging in online tissue subtyping and pathology with a direct prognostic impact, will speed up the implementation process. Focusing on the application of single-cell mass spectrometry imaging in digital pathology, the main requirements for the implementation of a new method are 1) robustness and repeatability, 2) speed, and 3) clear and objective interpretation without in-depth mass spectrometry knowledge. With this research all these requirements are met.

Although mass spectrometry based methods for identifying samples have been described in the literature, these methods rely on mass spectrometry analysis of a whole sample by lysis and processing of said sample. Therefore such methods lose any information about single cells and localization of cells within a sample, which may be relevant from a diagnostic point of view. As far as the applicant is aware, this is the first time mass spectrometry imaging is used to identify single cells in a sample.

The methods described herein can be used for identifying or characterizing one or more single cells. When used herein, the term identifying or characterizing a cell refers to assigning a cell type, phenotype or characteristic to the cell, for example by comparing it to reference cells. For example the method may be used to identify different cell types in a sample, or may be used to identify a tumor subtype in a sample or to distinguish between healthy cells and cells infected with a pathogen. The method may be used on specific samples, such as tumor samples to, for example, identify tumor stem cells or specific non tumorigenic cells such as immune cells in the tumor sample.

When used herein, the term “one or more single cells” in the context of identifying or characterizing refers to the method being able to identify a (one or more) specific single cell's identity or characteristic, as opposed to identifying the presence of one or more specific cells in a sample. For example, using traditional MS techniques a sample can be analyzed and the presence of one or more specific cell types in the sample can be inferred, however the information is lost which cell in the sample is the identified cell and the localization of said cell in the sample. Using the claimed method, for example an image is provided of the sample (e.g. a microscope image) allowing subsequent identification of each cell in the image, thereby providing a identity for each individual single cell and a correlation with its localization in the sample. Therefore in an embodiment the method further includes the step of providing an image of the sample and assigning or annotating the identified one or more single cells in the image.

When used herein the term sample may refer to any biological sample containing cells, such as but not limited to: organism or part thereof, tissue such as a biopsy, blood, serum, a mixture of cells on a surface from e.g. a fine needle aspirate, excrement, mucus, mother milk, cell culture, plant or part thereof. The cells in the sample may be animal cells including human cells, plant cells, fungal cells and/or bacterial cells.

Mass spectrometry imaging (MSI) is a technique used in mass spectrometry to visualize the spatial distribution of molecules, as molecular markers, metabolites, peptides or proteins by their molecular masses. After collecting a mass spectrum at one spot, the sample is moved to reach another region, and so on, until the entire sample is scanned. By choosing a peak in the resulting spectra that corresponds to the compound of interest, the MS data is used to map its distribution across the sample. This results in pictures of the spatially resolved distribution of a compound pixel by pixel. A mass spectrum is an intensity vs. m/z (mass-to-charge ratio) plot representing a chemical analysis. Hence, the mass spectrum of a sample is a pattern representing the distribution of ions by mass-to-charge ratio in a sample. It is a histogram usually acquired using an instrument called a mass spectrometer.

Suitable MSI methods are known and may for example be Secondary ion mass spectrometry (SIMS), Matrix-assisted laser desorption ionization (MALDI) or Desorption electrospray Ionization (DESI) based. Further suitable mass to charge ranges for use in the methods described here will be clear to the skilled person. For example if focusing more on metabolites a lower mass to charge range (e.g. 100 to 1300 m/z) may be used as opposed to when focusing on peptides, where a range from 1000 to 3500 m/z may be more suitable, while a range from 200 to 1300 m/z is more suitable for lipids. It is further envisioned that labelled or targeted profiles are measured, in which case the focus should be more on elemental profiles, e.g. 1 to 400 m/z. In the present method it is however preferable to not limit the method to specific types of molecules and use the whole mass to charge range available.

When used herein therefore the term single cell mass spectrometry imaging data refers to the mass over charge ratios (“MS peaks”) obtained from a single cell using MSI, which can thus be correlated to a single cell in a sample. This allows that in an image of the sample, the respective cell can be annotated as having a certain identity, and allows correlation of identity and localization of a single cell in a sample.

When used herein, the term recognition model refers to a model to identify one or more single cells in a sample based on reference data obtained from reference cell types. The model may be independently constructed and applied in the method according to the invention. Preferably the model is based in MSI data obtained from at least two distinct reference cell types, together referred to as the reference database, and compares the MSI data of the cell in the sample with the data in the reference database to calculate for each cell type in the reference database the probability that the cell in the sample is the same (similar as) the cell type in the reference database. Therefore, the term reference database when used herein may also refer to a predictive mathematical model based on reference data. The term reference data when used herein refers to mass spectrometry data, preferably MSI data, obtained from reference cells or one or more reference samples.

When used herein, the term cell type refers to any difference between two or more cells that may be of interest. For example, cell type may refer to its typical meaning of specific cell types in a tissue, organ or organism. The term may also refer to cells with a specific mutation, phenotype, genotype or a cell having a certain characteristic. Non limiting examples are cells with a certain mutation, subspecies or a pathogen, cell infected with a pathogen, tumor type or subtype, etc.

It is understood that using the methods described herein, a single cell in a sample may be analyzed or characterized, however the method may also be applied to multiple cells in the sample, such as 2, 3, 4, 5, 6, 7, 8, 9, 10, 20, 30, 40, 50, 100, 200, 300, 400, 500, 1000, 2000, 3000, 4000, 5000, 10000 or more cells in a sample. For example for the purpose of pathology or diagnosis it may be desirable to analyze or characterize many of the cells in the sample to obtain a better understanding of different cells present the sample.

When used herein, the term dimensionality reduction algorithm refers to any suitable algorithm that may be used to compare the mass spectrometry data obtained from the sample with the mass spectrometry data in the reference database. Suitable dimensionality reduction algorithms are known to the skilled person. Non limiting examples are Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbor Embedding (t-SNE). Therefore, in an embodiment of the invention, the dimensionality reduction algorithm is selected from Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbour Embedding (t-SNE).

It is appreciated that the methods described here may be performed using a single mass spectrum obtained for each cell type, however it is understood that accuracy and reliability may be improved by obtaining multiple, for example two, three, four, five or more mass spectra for each cell in the sample. Therefore in an embodiment of the invention, the single cell mass spectrometry imaging data obtained from the sample comprise for each single cell at least two, preferably three, four, five or more, spectra. In an embodiment, said two or more spectra in the single cell mass spectrometry imaging data obtained from the sample are obtained from distinct subcellular locations. Obtaining mass spectra from distinct subcellular locations allows for additional information which may be used by the algorithm to identify the cell. For example it allows to distinguish between cytosolic or nuclear localization of a compound (or combination of compounds).

The use of matrix-assisted laser desorption ionization (MALDI) MSI is further contemplated in the methods of the invention. MALDI MSI technique in which the sample, often a thin tissue section, is moved in two dimensions while the mass spectrum is recorded, although 3D MSI may also be used. Advantages, like measuring the distribution of a large amount of analytes at one time without destroying the sample, make it a useful method in tissue-based study. The matrix must absorb at the laser wavelength and ionize the analyte. Matrix selection and solvent system relies heavily upon the analyte class desired in imaging. The analyte must be soluble in the solvent in order to mix and recrystallize the matrix. The matrix must have a homogeneous coating in order to increase sensitivity, intensity, and shot-to-shot reproducibility. Minimal solvent is used when applying the matrix in order to avoid delocalization. One technique is spraying. The matrix is sprayed, as very small droplets, onto the surface of the sample, allowed to dry, and re-coated until there is enough matrix to analyze the sample. The size of the crystals depend on the solvent system used. Sublimation can also be used to make uniform matrix coatings with very small crystals. The matrix is placed in a sublimation chamber with the mounted tissue sample inverted above it. Heat is applied to the matrix, causing it to sublime and condense onto the surface of the sample. Controlling the heating time controls the thickness of the matrix on the sample and the size of the crystals formed. Particularly suitable methods and devices for sublimation of the matrix are described in WO 2020/078830 which is hereby incorporated in its entirety. The device described in WO 2020/078830 allows for a crystal size of the matrix in the sub 10 micron to sub 1 micron range, allowing for a substantially higher resolution of imaging. Therefore in an embodiment of the invention, the sample has a matrix with an average crystal size of at most 10 micron, preferably at most 1 micron.

A particular advantage of the methods described herein is the ability to perform on the fly analysis of cells in a sample. This is particularly useful for example during a medical procedure to obtain a quick diagnosis or analysis of a sample. It is therefore desirable that the sample is quickly processed and imaged. Therefore, in an embodiment of the invention, the time required to apply the matrix is at most five minutes per sample. Preferably the time needed is at most four minutes, more preferably at most three even more preferably at most two most preferably at most one minute. The inventors have demonstrated that processing and imaging of a sample within the minute range is feasible.

It is understood that the reference database is independently obtained and as such does not need to be generated every time the method of the invention is performed. Therefore in an embodiment of the invention, the reference database is independently obtained. In a further embodiment the reference database is independently obtained according to the method of third aspect of the invention.

When used herein, the term characteristic may refer to for example a tissue type, a cell type, a tumor type, a tumor subtype, a genetic aberration, a pathway activity, expression profile, a molecular characteristic, a morphological characteristic, immuno-histochemical profile, a viral infection, a bacterial infection or an infection with a pathogen which can be used to distinguish two different cells. Therefore, in an embodiment of the invention, the known characteristic or characteristics is on or more selected from a tissue type, a cell type, a tumor type, a tumor subtype, a genetic aberration, a pathway activity, expression profile, a molecular characteristic, a morphological characteristic, immuno-histochemical profile, a viral infection, a bacterial infection or an infection with a pathogen.

It is understood that the method of the invention may be used for diagnostic or pathology purposes. For example the method may be used to diagnose or characterize a tumor based on the information obtained from the individual cells present in the tumor, as further exemplified by the examples detailed below. This can be achieved by generating a reference database with different tumor cell types. For example in order to diagnose or characterize breast cancer, the reference database may be generated using different breast cancer cell lines. It is understood that for the reference database data can be obtained from representative samples but also from defined cell lines. It was found by the inventors that a particularly accurate model can be constructed by using data obtained from cell lines in the reference database, as the individual cell lines are uniform Therefore in an embodiment of the invention, the distinct cell types in the reference database are distinct cell lines. When used herein, the term cell line refers to a cell culture developed from a single cell and consisting of cells with a uniform genetic make-up.

The method may further also be used to distinguish between different infections with a pathogen such as a bacteria or virus. For this purpose, a reference database can be generated with cell lines infected with the different pathogens. Therefore in an embodiment of the invention, the distinct cell types in the reference database are distinct cell lines or cells infected with distinct pathogens or viruses. When used herein, the term pathogen may refer among others to algae, bacteria, viruses, viroids, protozoans, fungi (including yeasts), prions or parasitic worms.

It is understood that by decreasing the pixel size in the MSI method, a higher resolution can be obtained which is favorable for the methods used described herein. Preferably the pixel size is such that it does not exceed cell size, however more preferably the pixel size is such that for each cell is covered by at least two, more preferably at least three, four, five or more pixels. Therefore in an embodiment of the invention, the mass spectrometry imaging method uses a pixel size of at most 100 μm², preferably at most 64 μm², more preferably at most at most 36 μm², most preferably below 25 μm².

A particular advantage of the present invention is that it allows to identify a particular single cell in a heterogenous sample. This is particularly useful in the characterization or diagnosis of cancer in a sample. For example, blood comprises many different (healthy) cell types but may, in the case of leukaemia, also comprise tumorigenic cells. The present method allows to detect these tumorigenic among the plethora of healthy cells to diagnose the subject or characterize the sample based on the presence of the identified cells. Therefore, in an embodiment of the invention, the method is used to detect or identify a single cell type in a heterogenous sample, preferably wherein said single cell type is one of: a circulating tumor cell in a blood or serum sample; an immune cell in a tissue sample; or a tumor subtype or tumor stem cell in a tumor sample. Furthermore, because the present method allows for localization of the specific cell in the sample, the method may for example be used to determine the presence or distribution of immune cells in a tumor sample to determine the feasibility or success of an immune based cancer therapy.

In a second aspect the invention relates to a method of diagnosing a subject with cancer using the method as defined in the first aspect of the invention, wherein the sample is a tumor sample obtained from the subject, and wherein the diagnosis is based on the characteristic or characteristics from the reference cell which has or have been assigned to the cell in the sample, and wherein the two or more distinct cell types in the reference library are two or more distinct tumor cells, preferably two or more cancer cell lines. Therefore, the invention relates to a method of diagnosing a subject with cancer using a method for identifying one or more single cells in a sample, the method comprising: performing mass spectrometry imaging (MSI) on the cell in the sample to obtain single cell mass spectrometry imaging data from the cell in the sample; applying to the obtained single cell mass spectrometry imaging data from the cell in the sample a recognition model to identify the cell in the sample; wherein the recognition model comprises a reference database to which a dimensionality reduction algorithm has been applied, wherein the dimensionality reduction algorithm has classified the data from the reference database in classes representing distinct cell types, obtaining classified reference data, wherein the applying of the recognition model to the obtained single cell mass spectrometry imaging data from the cell in the sample comprises assigning a probability that the obtained single cell mass spectrometry imaging data from the cell in the sample belongs to a class within the classified reference database, and wherein the cell in the sample is identified based on the highest probability; wherein the reference database comprises single cell mass spectrometry imaging data, obtained by mass spectrometry imaging, from at least two distinct cell types with known and differing characteristics, wherein the single cell mass spectrometry imaging data in the reference database and the single cell mass spectrometry imaging data obtained from the sample comprise for each cell at least one spectra, wherein the sample is a tumor sample obtained from the subject, and wherein the diagnosis is based on the characteristic or characteristics from the reference cell which has or have been assigned to the cell in the sample, and wherein the two or more distinct cell types in the reference library are two or more distinct tumor cells, preferably two or more cancer cell lines.

When used herein, diagnosis may refer to identifying or characterizing a disease, e.g. identifying the presence of tumor cells or cells infected with a pathogen in a sample, or in a tumor sample or infected sample characterizing the tumor or pathogen type or subtype. In the case of breast cancer for example, the method may be used to distinguish between ER, PR and Her2 positive or negative tumor samples. Therefore, in an embodiment the tumor sample is a breast cancer sample and wherein the diagnosing comprises at least classifying the tumor sample as ER positive or negative breast cancer, PR positive or negative breast cancer and/or Her2 positive or negative breast cancer.

The method may further comprise providing a treatment suggestion or treatment step, wherein an optimal treatment strategy is suggested or a treatment is administered based on the diagnosis or characterization of the sample. Therefore in an embodiment of the invention, the method further comprises suggesting an optimal treatment strategy and/or administering a drug based on an optimal treatment strategy, wherein the optimal treatment strategy is based on the diagnosis obtained for the subject.

In a third aspect the invention relates to a method for constructing a recognition model for identifying one or more single cells in a sample, the method comprising the steps of: providing at least two samples each comprising distinct cell types; performing single cell mass spectrometry imaging on at least two cells in each sample in a predefined m/z range, wherein for each cell a minimum of one, preferably three, single cell mass spectrometry imaging datasets are obtained; building a database comprising for each cell line the single cell mass spectrometry imaging data within the predefined m/z range and the corresponding subcellular localization data; and constructing the recognition model by applying a dimensionality reduction algorithm to the reference database. The reference database constructed by this method finds use in the methods of the first and second aspect of the invention. In an embodiment the dimensionality reduction algorithm is selected from Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbor Embedding (t-SNE). In an embodiment the at least two samples comprising distinct cell types are at least two distinct cell lines.

It was found by the inventors that in order to achieve particularly accurate predictions and efficiency of the identification model, it is preferred that the recognition model is based on mass spectrometry imaging data obtained form single cells.

### DESCRIPTION OF THE FIGURES

FIG. 1: Experimental workflow from single cell preparation to digital pathology A. Cell preparation on poly-L-lysine coated ITO slides B. Sample sublimation and mass spectrometry imaging analysis C. Data analysis containing ROI selection for every single cell, determination of mean mass spectrum of every cell after RMS normalization, model building using PCA/LDA analysis and applying the generated method offline as well as online for pathological identification.

FIG. 2: Single cell images of 4 different breast cancer cells types measured on MALDI-TOF with spatial resolution of 5×5 μm². A. Optical microscopy images (10×) after mass spectrometry imaging B. Distribution of PC 36:1 in the different breast cancer cell types C. Detail of a single HCC1143 cell. The optical image (left) and detailed distribution of PC 36:1 and LPC 18:0.

FIG. 3: DDA identification of lipids measured on Orbitrap Elite. A. Full MS in positive mode and B. MS2 of 788.61 (PC 36:1). (A) and (B) both measured in MDA-MB-231 cell line C. Heat map of 79 identified lipids based on DDA analysis of single cells. Lipids identified are shown for 3 representative individual cells plotted per cell type.

FIG. 4: Classification models in AMX model builder (Waters) of A. different genetic phenotypes and B. cell types, based on single cell profiles obtained from timsTOF flex imaging experiments.

FIG. 5: Comparison of A. mean normalized mass profiles and B. bar diagram (top) and trend line (bottom) of relative abundances of 10 differentiating lipids according the cell type model of a single MDA-MB-231 cell after RMS normalization measured on different mass spectrometry imaging instruments (positive mode). Error bars indicate the standard deviation. 3 randomly chosen cell profiles were used in the plots.

FIG. 6: Offline automatic recognition of xenograft subtypes MDA-MB-231 (A-C) and MDA-MB-468 (D-E). Top row (A and D) is showing H&E staining (left), optical image of section (middle) and the distribution of identified cell types combined with the overlayed optical image (right). The distribution of cell types in separate images are shown in B and E. All images shown in A, B, D and E are based on timsTOF flex MALDI-1 data. Comparison of cell type recognitions measured on timsTOF flex MALDI-1 and Synapt G2-Si HDMS are show in C and F. All measurements show the correct identity taking the number of identified spectra into consideration.

FIG. 7: Example of the spatial distribution of all analyzed cell types. Scale bar represent 200 μm.

FIG. 8: Detailed cross validation table of A. different genetic phenotypes and B. cell subtypes

FIG. 9: Classification example of Xenograft according to the status of ER, PR, HER2

FIG. 10: Intensity box plot of top 10 differentiating lipids in a representative cell ROI. Error bars showing the difference within the ROI area. Dots next to the error bars represent the intensity in a single pixel scan.

### EXAMPLES

### Example 1—Materials & Methods

### Chemicals and Reagents

Methanol (LC-MS grade), chloroform (HPLC grade), ethanol (HPLC grade), norharmane (crystalline), red phosphorus (>99.99% purity), poly-L-lysine solution 0.1% (w/v) in H2O, ammonium formate, PBS, Neutral Buffered Formalin, eosin, hematoxylin and xylene were purchased from Sigma Aldrich (Zwijndrecht, The Netherlands) and used without further purification.

### Cell Preparation on Slides

Breast cancer cell lines were purchased and cultured in buffer as indicated in supplemental information table 1. Indium Tin Oxide (ITO, CG-40 IN-S115, Delta Technologies, USA) glass slides are coated with poly-L-lysine (20 μl of 1:1 dilution in water). Slides were washed with water before placing them in a 60 mm petri dish with the conductive side facing up. Approximately 10⁶ cells (˜1.5*10⁵ cells/mL) were added into the petri dish and incubated overnight at 37° C. with 5% CO₂. Media was removed and slides were washed twice with 1×PBS. 10% Neutral Buffered Formalin was added for 10 minutes. Slides were washed twice with 50 mM ammonium formate and twice with Millipore water and dried under a gentle nitrogen stream.

### Breast Cancer Xenograft Models

To generate tumors, MDA-MB-231 or MDA-MB-468 (1.0*10⁶) cells were resuspended in 50 μl Matrigel™ Basement Membrane Matrix (BD) and were injected subcutaneously unilaterally into the flank of female Crl:NU-Foxn1^{nu} mice. When tumors were palpable, tumor volume was assessed by measuring the tumor in three dimensions using a Vernier caliper and using the formula a*b*c*π/6, where a, b, and c are orthogonal diameters of the tumor, each corrected for the thickness of the skin (0.5 mm). At a tumor volume of ca. 200 mm³, tumors were excised and snap-frozen.

### Sample Preparation for Mass Spectrometry Imaging

Tissue sectioning (12 μm, at −20° C.) was performed on fresh-frozen tissues using a Leica CM1860 UV cryotome (Wetzlar, Germany). All samples were stored at −80° C. before sectioning. MDA-MB-231 xenografts were embedded in gelatin prior to freezing and sectioning. Sublimation of 80 mg norharmane at 140° C. for 180 seconds was performed using HTX sublimator (HTX Technologies, USA). Sample preparation of xenografts were the same for offline and online recognition.

TimsTOF fleX (MALDI-1-MSI & MALDI-2-MSI)

Unless otherwise noted, MALDI, MALDI-2, and ion mobility (IMS)-mass spectrometry imaging was performed on the timsTOF flex MALDI-2 (Bruker Daltonics, Germany) in positive mode with 50 laser shots per pixel and interlaser pulse delay of 10 μs. Transfer settings were 350 Vpp (funnel 1 RF), 400 Vpp (funnel 2 RF), 600 Vpp (multipole RF). Focus Pre TOF transfer time was set at 90 us and prepulse storage at 10 μs. Quadrupole ion energy was 5.0 eV with low mass 300 m/z. Collision cell energy was 10.0 eV with collision RF at 200 Vpp. Positive ion mode MALDI-MSI measurements were performed. All spectra were recorded using 1 kHz laser repetition rates with 250 laser shots accumulated at each pixel. The average acquisition rate was 20 pixels per second over an m/z range between 200 and 1200 using a 5×5 μm² pixel size. Calibration of the instrument was carried out prior to every measurement with red phosphorus.

### Synapt

A Waters Synapt G2-Si HDMS system equipped with a prototype uMALDI source and provided with a Nd:YAG laser (Waters Corporation, UK) was used for online recognition experiments. For more detailed information about the uMALDI source, see Barre et al., Data acquiring was performed using MassLynx version 4.1 and HDImaging version 1.5 software (Waters Corporation). For online recognition, our built model in AMX model builder was loaded into AMX recognition software that was coupled to the data acquisition file. All measurements were performed in sensitivity mode with a scan rate of 1.0 s per scan, trap CE of 4, and transfer CE of 2, 1000 Hz laser repetition rate and mass range 300-1200 m/z in positive ion mode. The instrument was calibrated with red phosphorus for positive ion mode before each measurement. The spatial resolution was 30×30 μm².

### Orbitrap-Elite DDA

Data was acquired using a MALDI/ESI Injector (Spectroglyph LLC, Kennewick, USA) coupled to an Orbitrap Elite™ Hybrid Ion Trap-Orbitrap Mass Spectrometer (Thermo Fisher Scientific GmbH, Bremen, Germany). The MS1 data was acquired at a nominal mass resolution of 240,000 (FWHM @ m/z 400) across m/z 200-1300 while MS/MS data was acquired in parallel using the ion trap with an isolation width of 1 Da, activation Q was 0.170 and a normalized collision energy of 30 (manufacturer units).

### Rapiflex

Bruker RapifleX MALDI Tissuetyper™ time-of-flight (TOF) instrument equipped with a smartbeam laser (Nd:YAG 355 nm) operating at 5,000 Hz with 500 laser shots accumulated at each pixel was employed for MALDI-MSI. MALDI analyses were performed in the reflector positive mode in mass range 200-1200 m/z and sample rate 1.25 GS/s. Calibration was made in positive mode using Red Phosphorus. The instrument was used at 5×5 μm² pixel size.

### Data Analysis

After acquisition, data was imported and analyzed in MassLynx version 4.1 and HDImaging version 1.5 software (Waters Corporation), SCILS Lab MVS (Version 2020b Premium 3D, build 8.01.12082, Bruker Daltonics), FlexImaging (Version 5, Bruker), XCalibur (version 4.2.28.14, Thermo Scientific Scientific) and LipostarMSI (version 1.10b17, Molecular Horizon). Figures were prepared in Abstract Model builder (AMX, version 0.9.2092.0 [beta], Waters), SCILS Lab, mMass (5.5.0, www.mmass.org), and the Office 2016 software (Microsoft). LIPID MAPS Structure Database (http://lipidmaps.org) and ALEX123 lipid database (http://alex123.info/ALEX123/MS.php) were employed for molecular identification. Full details, including the lipid identification workflow, are described in Ellis et al.¹³. The offline recognition model was built in SCILS Lab. Here, cells were randomly assigned to a training set and a test set (two-third and one-third of cells, respectively). For online recognition, AMX Model builder and Recognition software (Waters, v 1.1.1966.0) were used.

### Staining

Hematoxylin and eosin (H&E) staining was performed on the same sections used for MALDI-MSI experiments. Following MALDI experiments, ITO slides were first dipped in a 70% EtOH solution for 5 min in order to remove the residual matrix. Then, the H&E staining was carried out. Briefly, slides were hydrated in water for 1 min followed by hematoxylin staining for 3 min, washed under running tap water for 3 min, stained with eosin for 30 sec and washed under running tap water for 3 min. Slides were then immersed in 100% EtOH for 1 min, transferred to xylene for 2 min, carefully covered with a coverslip, and dried at RT. The optical images were acquired at high resolution using the Leica AperioCS2 scanner with Aperio ImageScope (version 12.4.3.5008) software (Leica Biosystems Imaging, Nussloch, Germany).

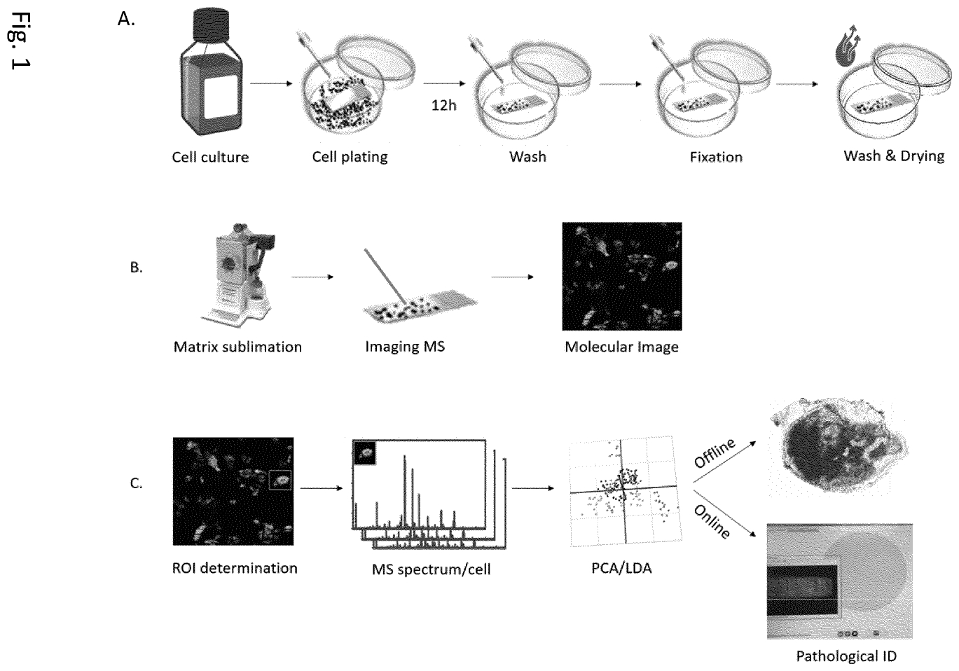

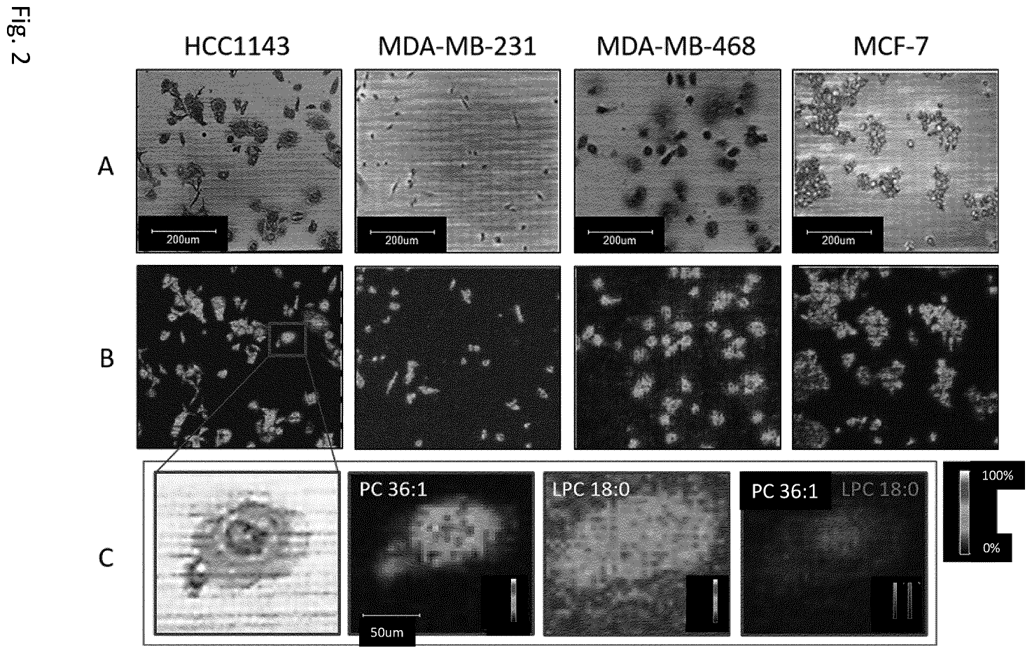

An overview of the full experimental workflow is shown in FIG. 1.

### Example 2—Mass Spectrometry Imaging of Single Cells

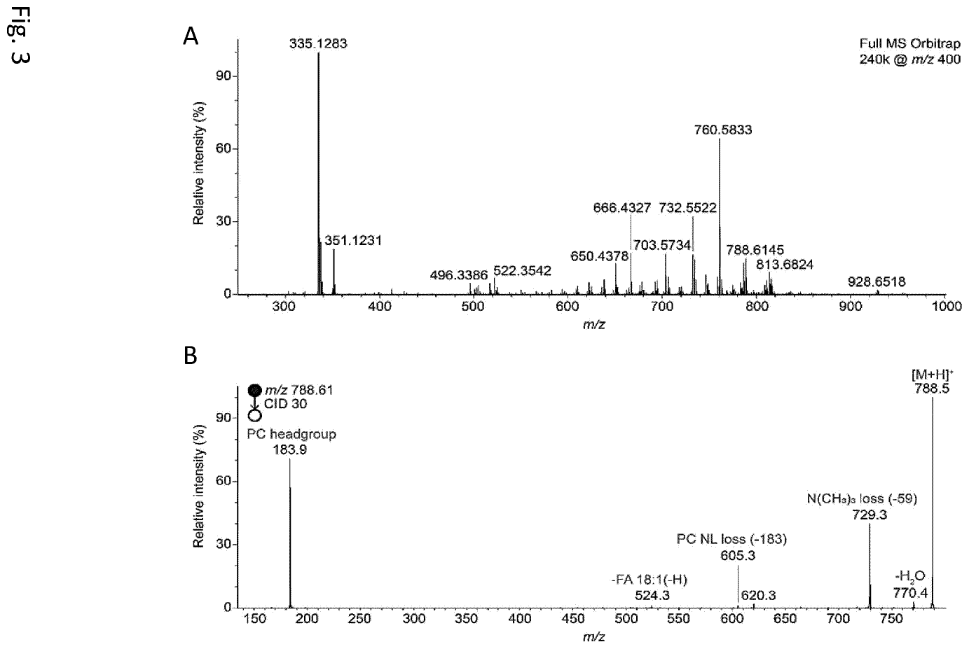

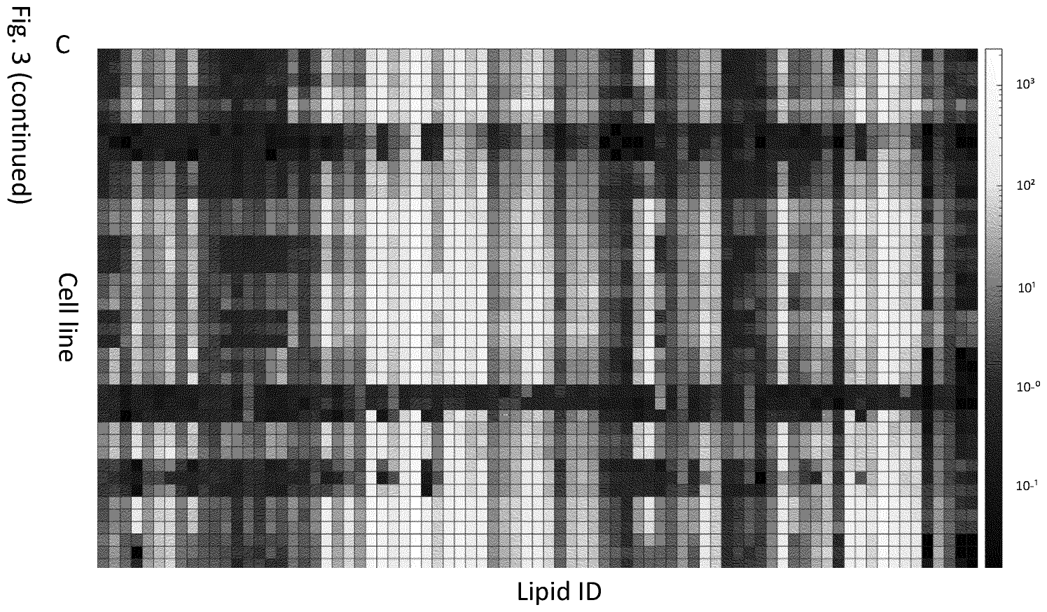

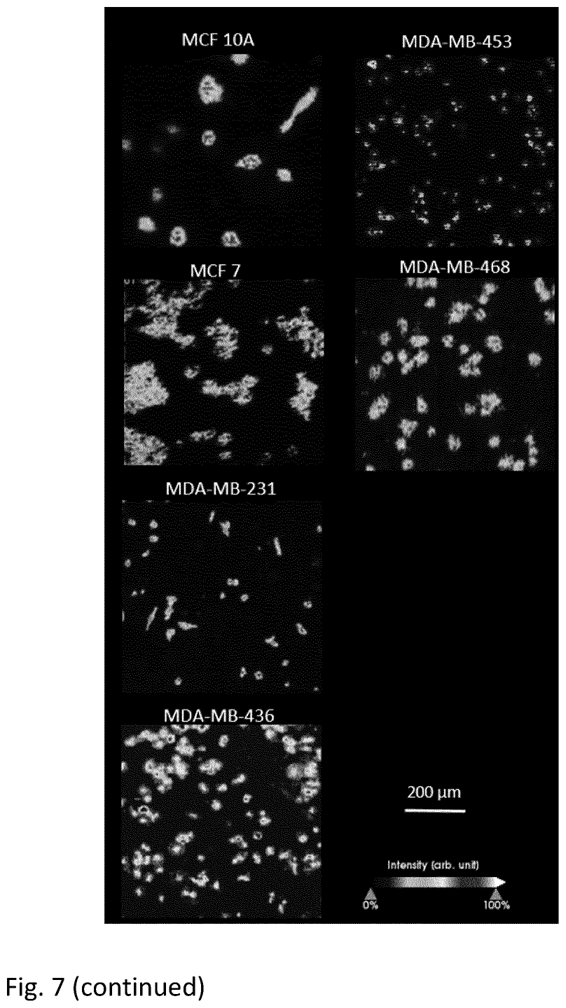

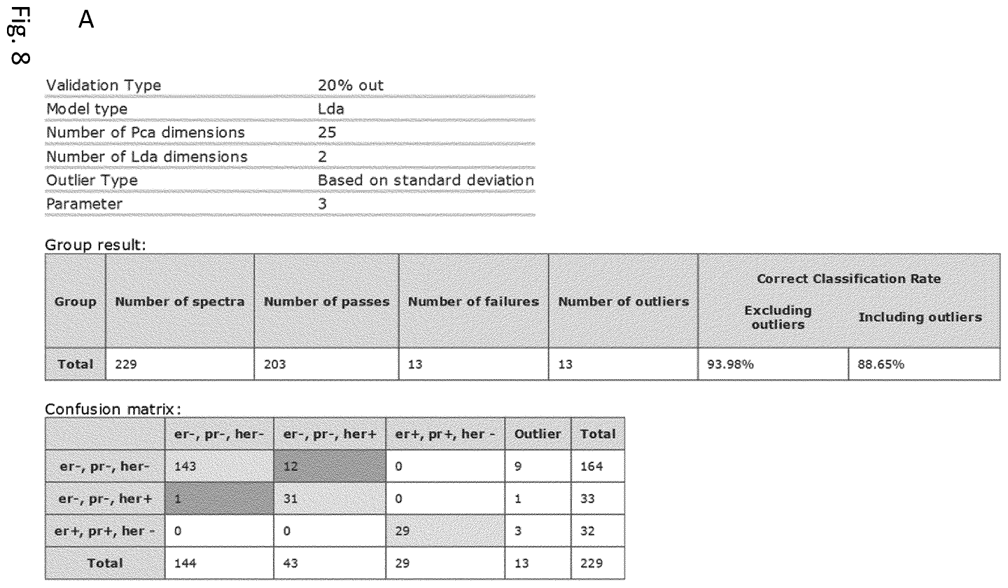

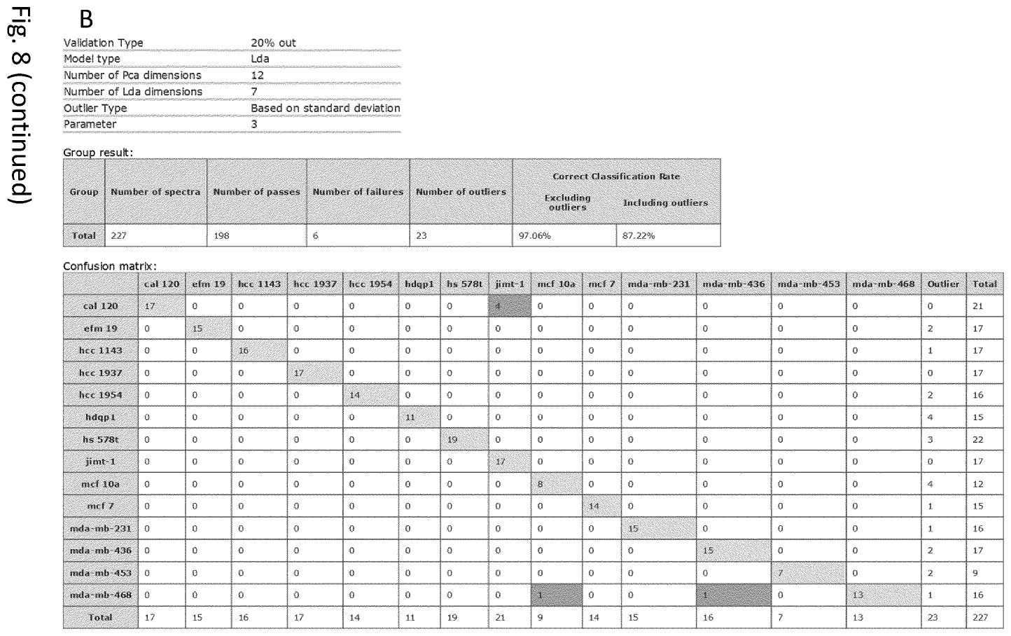

Fourteen different breast cancer cell lines were grown on poly-L-lysine coated ITO slides and MALDI-MSI data were acquired in positive mode (FIG. 7) to study the molecular composition of single breast cancer cells. The repeatability of the method was tested by comparing the molecular information of different cell cultures and different ITO sample plates of the same cell type. Moreover, the same samples were repeatedly measured on different days in order to rule out inter-day equipment differences. Before and after imaging, cell distribution, density, and shape were checked with light microscopy. All single cell types had a diameter between 20 and 150 μm. A 5×5 μm² pixel size allowed the acquisition of a minimum of 4 spectra per single cell. This high spatial resolution made it possible to visualize the intracellular distribution of compounds (FIG. 2). Full single cell spectra were analyzed by manually assigning the ROI for every cell and determining the mean spectrum of this ROI after Root Mean Square normalization. For every cell type, 3 different slides were measured on different days and in every measurement 3-5 individual cells were randomly selected as ROI. We were able to acquire in total 229 single cell spectra of 14 different cell types in order to build the recognition model with this approach. This repeatable single cell imaging method opens the possibility to discover cell type related molecular differences on a single cell level (inter- as well as intracellular).

### Example 3—Lipid Identification at the Single Cell Level

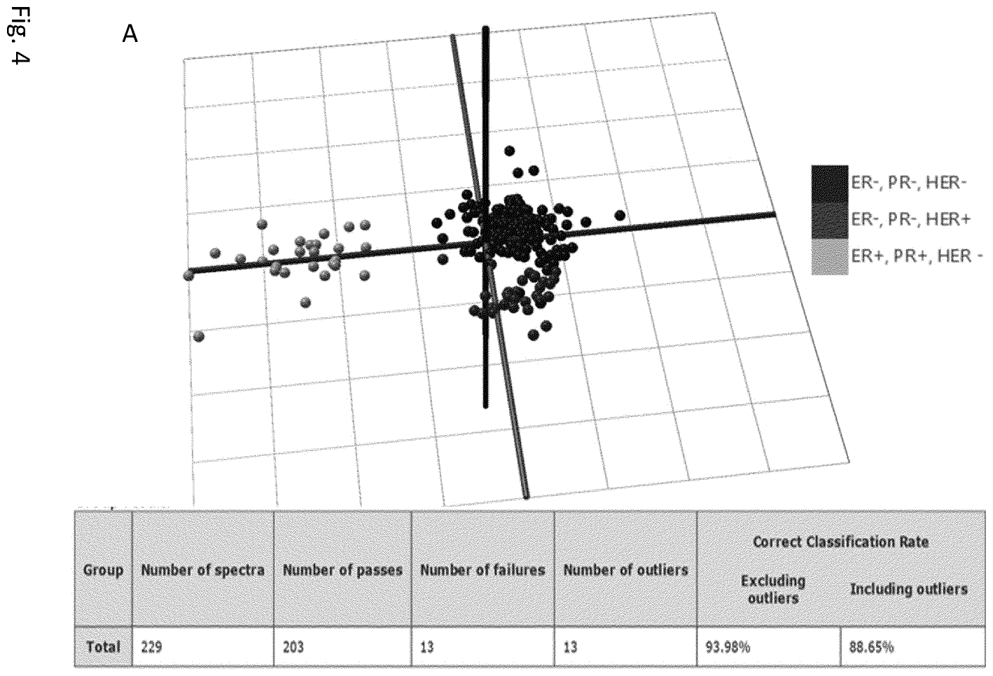

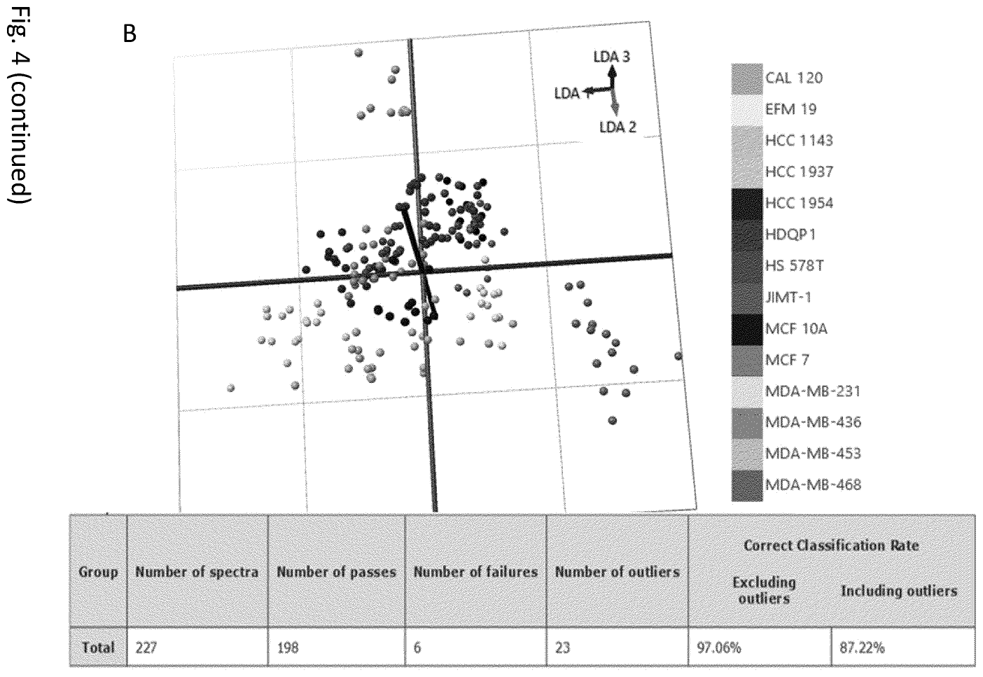

Automated, parallel mass spectrometry imaging and structural identification of lipids were obtained by Data Dependent Acquisition as previously described Ellis et al.¹³. These measurements were acquired on the same single cell slides as used in the imaging experiments and allowed the identification of 79 lipids present in all 14 cell lines. Crucial to the success of this DDA approach is a sufficiently large number of cellular pixels to be present in the image. Here we acquired minimal 10 (MS) and 8 (MS/MS) pixel scans per identified mass in a single data set. Identities confirm earlier described lipids found in cell pellet extractions analyzed with UPLC-QTOF-MS and in MSI data on MDA-MB-231 xenografts. This means our single cell MSI method is representative and is comparable to earlier described research. FIG. 3 shows an example of a single cell spectrum (A) and the MS2 spectrum (B) of 788.61 (PC 36:1). The heat map of 79 identified lipids (FIG. 3C) already indicates that every cell type has its own specific lipid profile, meaning different ratios of the same identified lipids, making a distinction between genetically different breast cancer cell types possible.

### Example 4—Recognition Models

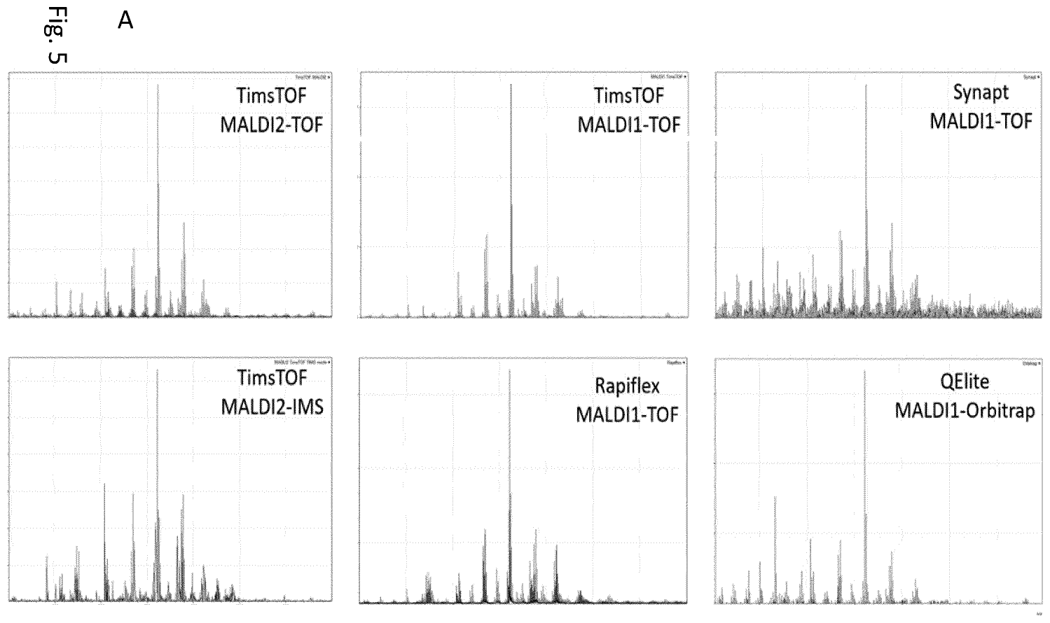

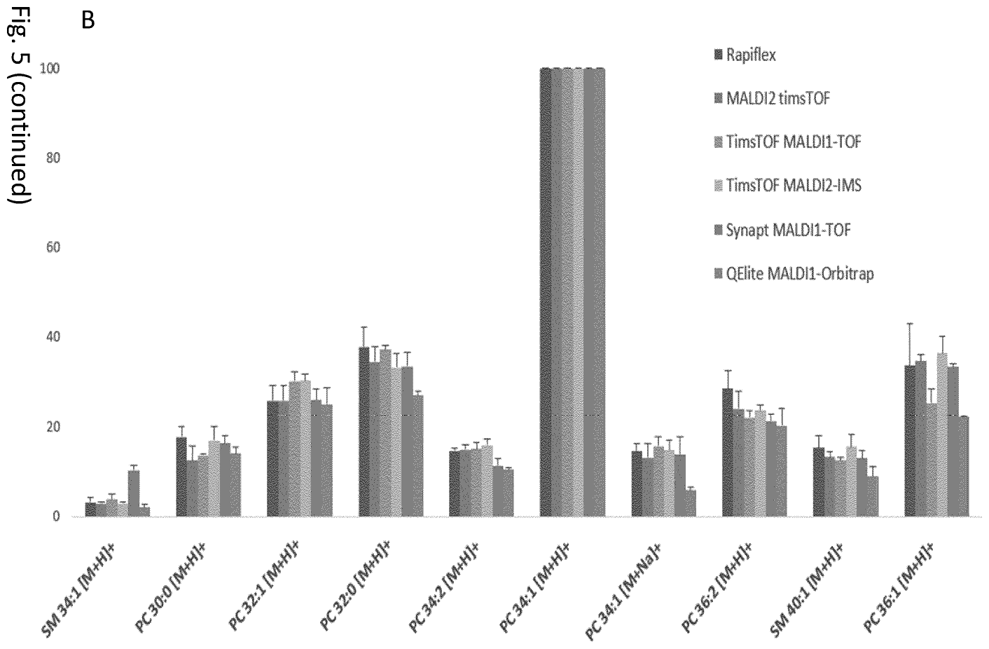

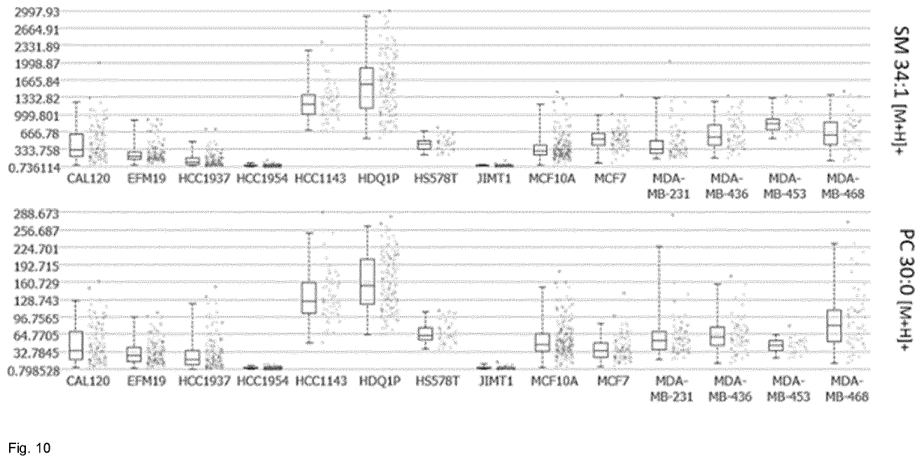

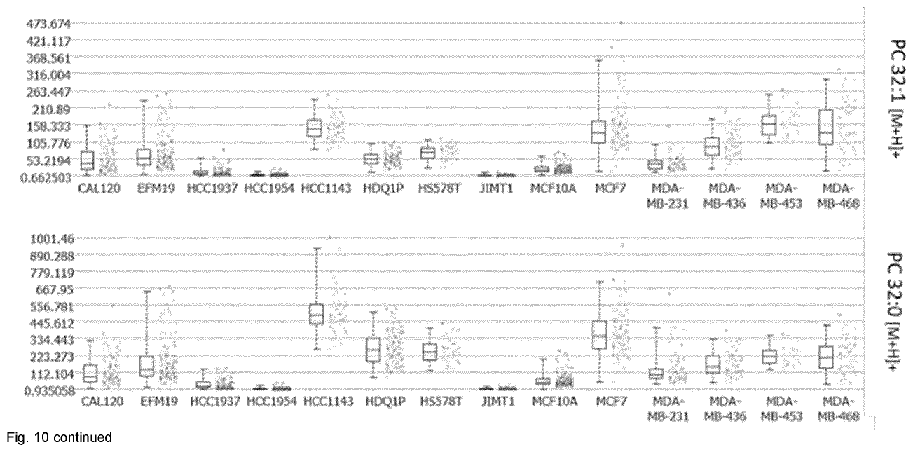

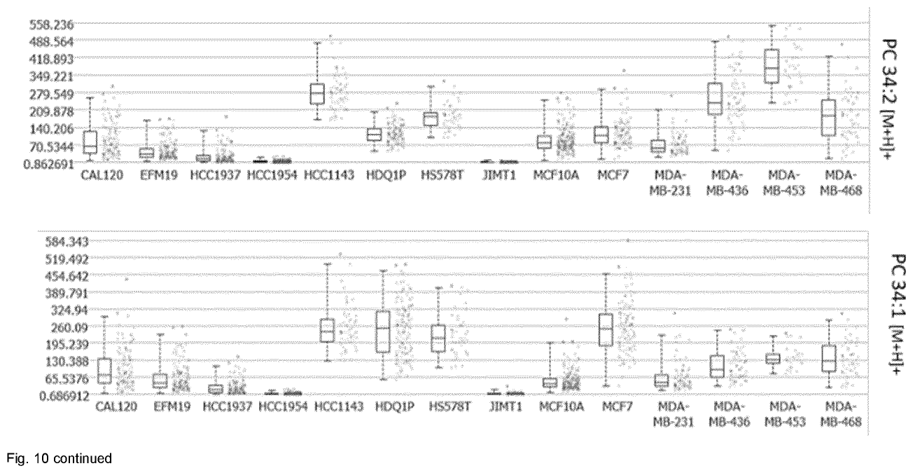

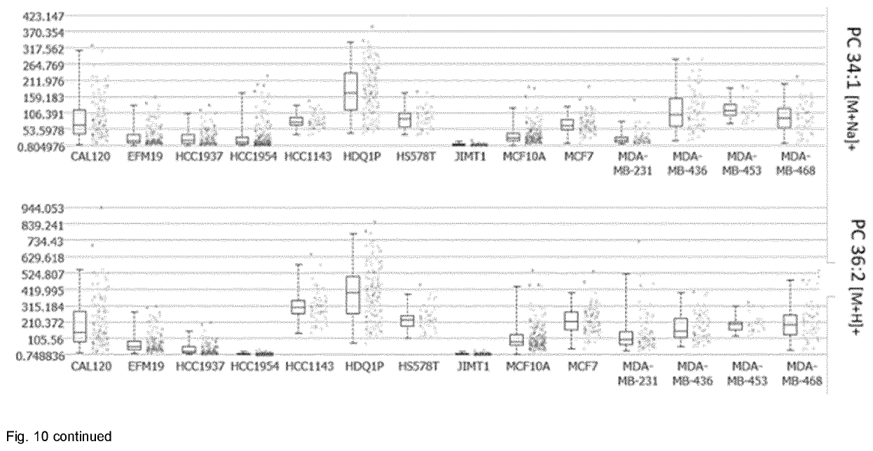

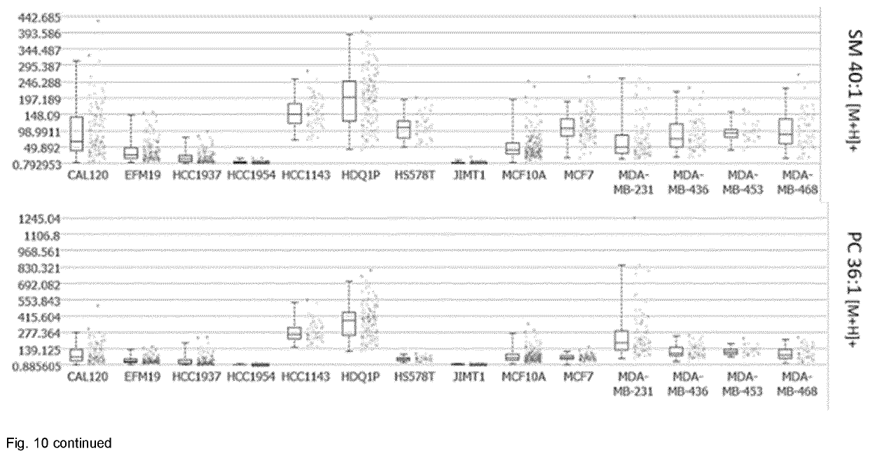

In general, breast cancer subtyping, according to the status of ER, PR, HER2, is considered to be a predictor of therapy response and survival prognosis of a patient. Indeed, tumor types with ER−, PR− and HER2− are described to be the most aggressive with the lowest survival rate. Patients with a hormone-receptor-positive tumor often clinically benefit from receiving hormonal therapies, which target the ER signalling pathway. Since it is previously described that lipid expression patterns are directly linked to estrogen receptor expression rates, we hypothesize that the lipid molecular profiles can be used to build a model that reveals the ER, PR, and HER2 status. As shown in FIG. 4A, we were able to separate the 3 groups (triple negative, HER2+, and ER+PR+) with PCA/LDA analysis based on a total of 229 single cell mass spectra between 600-950 m/z. It is important to notice that the model is built on the full mass profile within this mass range, thus not only using the identified lipid ratios. This model has a classification rate of 93.98% (excluding outliers) and 88.65% (including outliers) (FIG. 10) indicating it can be extremely valuable as reliable diagnostic but also therapy prognosis tool in breast cancer.

We also investigated whether we could make an even more detailed recognition model that is able to differentiate between cell type. A cell type classification model was built using PCA/LDA analysis based on the 229 single cell spectra acquired with timsTOF flex MALDI-2 in positive mode (FIG. 4B). A mass range from 600-950 m/z was used with a binning of 0.2 m/z. Cross-validation with 20% out and standard deviation 3 showed an excellent classification with 97.55% excluding outliers and 86.90% including outliers (FIG. 8B). Based on the mass and loading plots of the cell type recognition model and after DDA analysis, the 10 most prominent differentiating m/z values were all identified as lipids. Plots of the single cell intensities of these 10 lipids confirmed a specific profile for every cell type identity (FIG. 10).

In order to be able to use these recognition models as standard diagnostic and prognostic tools, there are two requirements that need to be fulfilled: 1) cell profiles are preferably MALDI-instrument independent so cell type visualization can be carried out based on our presented models without the need of separate models for every MALDI-instrument type; 2) cell profiles need to have in-tissue relevance in order to be able to recognize the correct cell type in a clinical relevant environment, namely resected patient tissue samples.

### Example 5—Comparative Analysis of Model Performance on Different MADLI-MSI Instruments

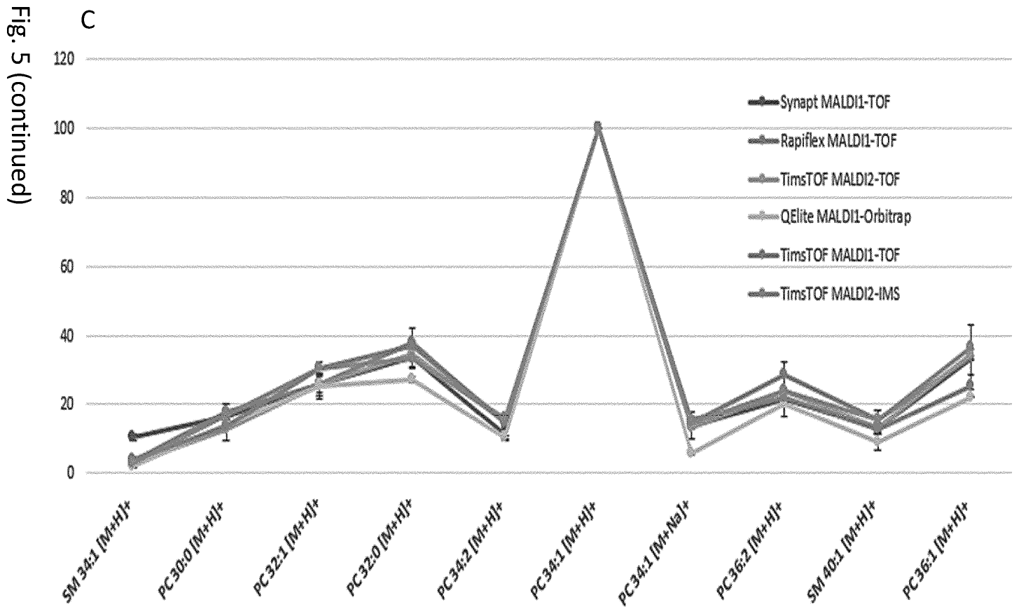

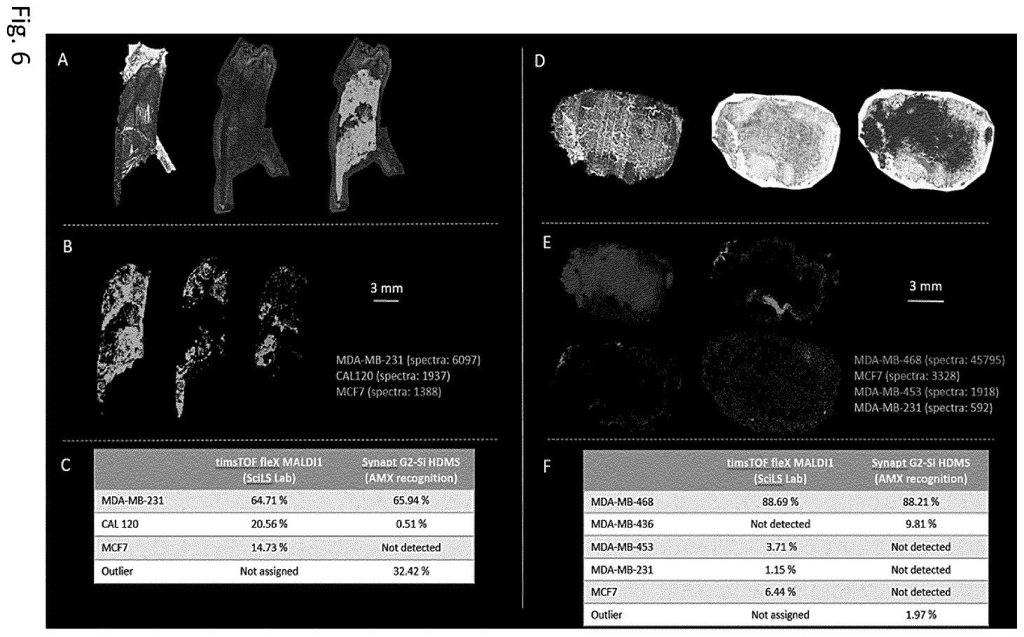

In order to build single cell, molecular models, we are facing two main challenges: 1) subcellular level spatial resolution is required with a minimum of 3 pixels per cell meaning that for cells of 20 μm diameter, we need a pixel size of 5 μm and 2) this high spatial resolution should be combined with a high sensitivity mode in order to acquire as much molecular information as possible in a broad m/z range. At the moment, these two requirements can be found in timsTOF flex MALDI-2. Indeed, it was described that timsTOF flex MALDI-2 technology increases the sensitivity by about 3 orders of magnitude²⁰⁻²². However, since our aim is to develop a general broadly applicable single cell type recognition method that can preferably be used for any MALDI-instrument, we investigated whether the acquired timsTOF flex MALDI-2 single cell profiles (and accompanying recognition models) are comparable to spectra acquired with other MALDI-MSI techniques (Time-Of-Flight and orbitrap). The same samples were measured on timsTOF flex MALDI-1 mode (Bruker Daltonics), timsTOF flex TIMS mode (Bruker Daltonics), Rapiflex (Bruker Daltonics), MALDI-LTQ Orbitrap Elite (Thermo Fisher Scientific), and Synapt G2-Si HDMS (Waters). Single cell MDA-MB-231 spectra (n=3) acquired on the different instruments were normalized against the most abundant differentiating lipid (PC 34:1). These ratios were plotted in a bar diagram with error bars representing the standard deviation. As shown in FIG. 5, we found comparable ratios of the 10 most differentiating lipids according the cell type model. Statistical analysis on these ratios was carried out using single factor ANOVA. The ratios showed F values below the critical F value of 3.105, confirming the hypothesis there is no significant difference between the profiles. Moreover, the trend line clearly indicates the comparable pattern for these lipids for the different MALDI systems. This indicates that the model based on the timsTOF flex MALDI-2 TOF spectra might also be applicable to other MSI equipment tested.

### Example 6—Offline Digital Pathology

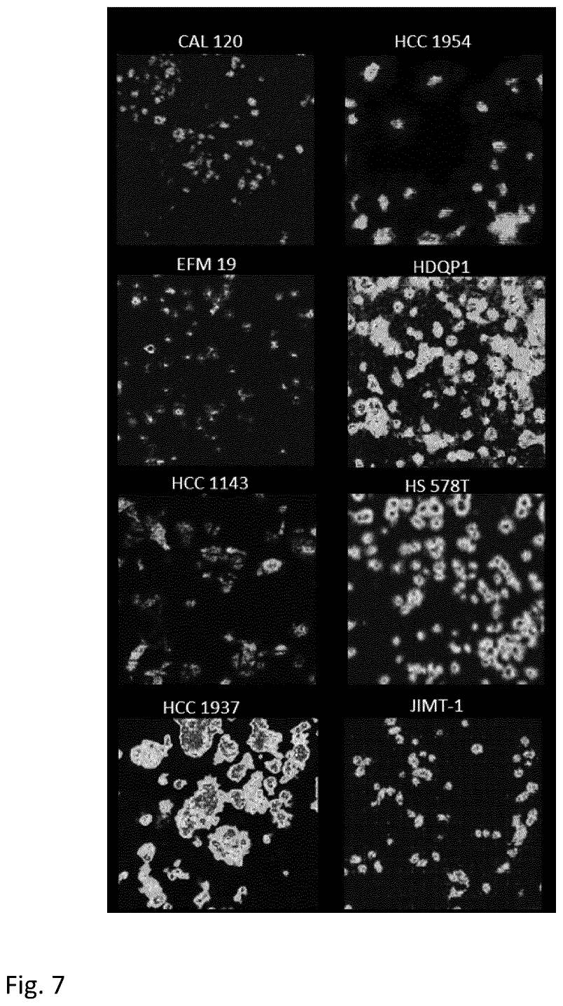

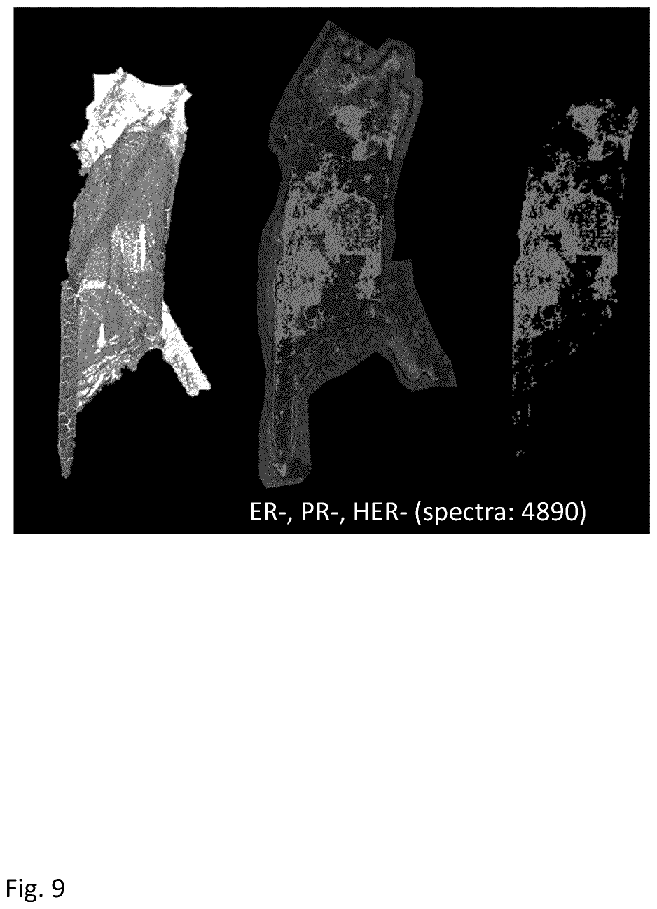

Cell profiles acquired on in vitro cultured cell lines need to have in-tissue relevance in order to be able to recognize the correct cell type in a tissue. To test this clinical validity of our cell type model, we assessed the model offline on xenograft tissue samples of MDA-MB-231 and MDA-MB-468. Using timsTOF flex, tissue samples were measured with 5×5 μm² spatial resolution. The acquired imaging data were offline processed using the cell type or genetic phenotype recognition model in SCILS Lab. Every 5 μm spot was automatically checked and classified according to the recognition model. FIG. 6 and FIG. 9 show that in both xenografts types, the main recognized cell type and genetic phenotype fits the original classification. This demonstrates the great sensitivity and high special resolution of the timsTOF flex MALDI-2 instrument. However, it is not compatible with any online recognition software that can directly be used during a measurement. Since Waters equipment is compatible with online recognition, this opens up the possibility for ‘on-the-fly’ tissue typing at cellular spatial resolution. Our comparative off-line study showed that the molecular single cell profiles of timsTOF flex and Synapt G2-Si HDMS are comparable (FIG. 5). Therefore, we expected that our timsTOF flex model is also applicable to Synapt G2-Si HDMS measurements on xenografts. In order to test this assumption, we compared MDA-MB-231 and MDA-MB-6468 xenograft measurements on timsTOF flex and Synapt G2-Si HDMS equipment, both measured with 30×30 μm² pixel size. AMX recognition software was run in post processing mode classifying every scan of the Synapt G2-Si HDMS measurement. SCILS Lab was used to classify the timsTOF flex data. The classification not only resulted in correctly identifying MDA-MB-231 and MDA-MB-468 as main cell type for both measurements, it also showed comparable percentages of the main cell type in the timsTOF fleX and Synapt G2-Si HDMS measurements (FIG. 6). Two other cell types (CAL120 and MCF7) were recognized in the MDA-MB-231 xenograft by SciIL Lab based on timsTOF flex data. These cell types are most probably a consequence of the SciLS Lab recognition system. The major difference between AMX recognition software and SciIS Lab is the fact that AMX allows outliers. SciLS Lab on the other hand forces all data points into one of the 14 cell classes of the model. If we consider the CAL120 and MCF7 classified scans as outliers, there is an outlier percentage of 35.29%. This is comparable to the outlier rate of 32.42% indicated by AMX recognition software. These outliers could be necrosis, background, gelatin, etc. In xenograft MDA-MB-468, all other cell types recognized are blow 10% and can most probably be considered as recognition errors.

In conclusion, these results confirm our statement that the recognition model is MALDI-TOF-instrument independent and shows that our cell type recognition model is applicable to xenograft samples, leading us to the ultimate goal: online digital pathology.

### Example 7—Online Digital Pathology

Ultimately, our model can be used in tissue for direct online cell type recognition. This would provide the pathologist instant molecular and prognostic information, possibly leading to better patient care. In order to test this, our built AMX model was loaded into AMX online Recognition Software (Waters) and MDA-MB-231 xenografts were run on Synapt G2-Si HDMS. As shown in the video (supplemental information), we were able to directly and online identify the cell type for every single laser spot. Whenever there is no tissue in the laser spot, the system recognizes this as ‘outlier’. This test shows that we are able to reach the ultimate goal of online ‘on-the-fly’ cell type recognition using mass spectrometry imaging. Moreover, this confirms our earlier findings that our models, based on acquired mass spectrometry imaging profiles from cultured single cells, are able to recognize the correct cell types in tissue samples. With these remarkable results, we are the very first to show direct ‘on-the-fly’ tissue typing based on single cell molecular IMS databases.

### Example 8—Online Recognition on Xenograft Sample

MDA-MB-231 Xenograft section was measured with Synapt G2-Si HDMS in positive mode at 1 scan per second. AMX software with our cell type recognition model was loaded. In every scan, the cell type is identified ‘on-the-fly’. When no cell type is recognized, the system output is ‘outlier’. The cell type is correctly identified on the tissue. Also outlier is correctly given in an area without tissue. We are the first to show ‘on-the-fly’ cell type identification using MALDI-TOF mass spectrometry imaging.

TABLE 1

| | | Code | | Medium to culture | | | | | | |
|---|---|---|---|---|---|---|---|---|---|---|
| Cell Line | Origin | (Provider) | Provider | cells at CP | ER | PR | HER2 | BRCA1 | Subtype | Tumor |
| CAL-120 | human | ACC 459 | DSMZ | 90% DMEM + 10% FBS | − | − | − | WT | TNB | AC |
| EFM-19 | human | ACC 231 | DSMZ | 90% RPMI 1640 + 10% FBS | + | + | − | WT | LA | IDC |
| HCC-1143 | human | ACC 517 | DSMZ | 80% RPMI + 20% FBS | − | − | − | WT | TNA | DC |
| HCC-1937 | human | ACC-513 | DSMZ | 90% RPMI 1640 + 10% FBS | − | − | − | MU | TNA | DC |
| HCC-1954 | human | CRL-2338 | ATCC | 90% RPMI 1648 + 10% FBS | − | − | + | WT | H | DC |
| HDQ-P1 | human | ACC 494 | DSMZ | 90% DMEM + 10% FBS | − | − | − | MU | TNB | IDC |
| HS-578T | human | ACC 781 | DSMZ | 90% DMEM + 10% FBS | − | − | − | WT | TNB | IDC |
| JIMT-1 | human | ACC 589 | DSMZ | 90% DMEM + 10% FBS | − | − | + | WT | H | DC |
| MDA-MB-436 | human | HTB-130 | ATCC | 90% DMEM + 10% FBS | − | − | − | MU | TNA | AC |
| MDA-MB-231 | human | ACC-732 | DSMZ | 90% DMEM + 10% FBS | − | − | − | WT | THB | AC |
| MDA-MB-453 | human | ACC-65 | DSMZ | 90% DMEM + 10% FBS | − | − | + | WT | H | AC |
| MDA-MB-468 | human | ACC-738 | DSMZ | 90% DMEM + 10% FBS | − | − | − | WT | TNA | AC |
| MCF-7 | human | HTB-22 | ATTC | 90% DMEM + 10% FBS | + | + | − | WT | LA | IDC |
| MCF10A | human | CRL-10317 | ATTC | 90% DMEM + 10% FBS | − | − | − | WT | luminal | epithelium |
| | | | | | | | | | ductal | |
| | | | | | | | | | gland | |

ER = oestrogen receptor
PR = progesterone receptor
HER = human epidermal growth factor receptor 2
WT = wild type
ND = not decided
MU = mutated
L = luminal
H = Her2
IDC = invasive ductal carcinoma
AC = adenocarcinoma
DC = ductal carcinoma
InfDC = inflammatory ductal carcinoma

1. A method for identifying one or more single cells in a sample, the method comprising:
performing mass spectrometry imaging (MSI) on the one or more single cells in the sample to obtain single cell mass spectrometry imaging data from the one or more single cells in the sample;
applying to the obtained single cell mass spectrometry imaging data from the one or more single cells in the sample a recognition model to identify the one or more single cells in the sample;
wherein the recognition model comprises a reference database to which a dimensionality reduction algorithm has been applied,
wherein the dimensionality reduction algorithm has classified the data from the reference database in classes representing distinct cell types, obtaining classified reference data,
wherein the applying of the recognition model to the obtained single cell mass spectrometry imaging data from the one or more single cells in the sample comprises assigning a probability that the obtained single cell mass spectrometry imaging data from the one or more single cells in the sample belongs to a class within the classified reference database, and
wherein the cell in the sample is identified based on the highest probability;
wherein the reference database comprises single cell mass spectrometry imaging data, obtained by mass spectrometry imaging of single cells, from at least two distinct cell types with known and differing characteristics,
wherein the single cell mass spectrometry imaging data in the reference database and the single cell mass spectrometry imaging data obtained from the sample comprise for each cell at least one spectra.

2. The method according to claim 1, wherein the dimensionality reduction algorithm is selected from Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbor Embedding (t-SNE).

3. The method according to claim 1, wherein the single cell mass spectrometry imaging data in the reference database and the single cell mass spectrometry imaging data obtained from the sample comprise for each single cell at least two, preferably three, four, five or more, spectra.

4. The method according to claim 3, wherein said two or more spectra in the reference database and the single cell mass spectrometry imaging data obtained from the sample are obtained from distinct subcellular locations.

5. The method according to claim 1, wherein the sample has a matrix with an average crystal size of at most 10 micron, preferably at most 1 micron.

6. The method according to claim 5, wherein the time required to apply the matrix is at most 5 minutes per sample.

7. The method according to claim 1, wherein the reference database is independently obtained.

8. The method according to claim 1, wherein the known characteristic or characteristics is on or more selected from a tissue type, a cell type, a tumor type, a tumor subtype, a genetic aberration, a pathway activity, expression profile, a molecular characteristic, a morphological characteristic, immuno-histochemical profile, a viral infection, a bacterial infection or an infection with a pathogen.

9. The method according to claim 1, wherein the distinct cell types in the reference database are distinct cell lines cells infected with distinct pathogens or viruses.

10. The method according to claim 1, wherein the mass spectrometry imaging method uses a pixel size of at most 100 μm², preferably at most 64 μm², more preferably at most at most 36 μm², most preferably below 25 μm².

11. The method according to claim 1, wherein the method is used to detect or identify a single cell type in a heterogenous sample, preferably wherein said single cell type is one of
a circulating tumor cell in a blood or serum sample;
an immune cell in a tissue sample; or
a tumor subtype or tumor stem cell in a tumor sample.

12. A method of diagnosing a subject with cancer using the method as defined in claim 1, wherein
the sample is a tumor sample obtained from the subject, and
wherein the diagnosis is based on the characteristic or characteristics from the reference cell which has or have been assigned to the cell in the sample, and
wherein the two or more distinct cell types in the reference library are two or more distinct tumor cells, preferably two or more cancer cell lines.

13. The method according to claim 12, wherein the tumor sample is a breast cancer sample and wherein the diagnosing comprises at least classifying the tumor sample as ER positive or negative breast cancer, PR positive or negative breast cancer and/or Her2 positive or negative breast cancer.

14. The method according to claim 12, wherein the method further comprises suggesting an optimal treatment strategy and/or administering a drug based on an optimal treatment strategy, wherein the optimal treatment strategy is based on the diagnosis obtained for the subject.

15. Method for constructing a recognition model for identifying one or more single cells in a sample, the method comprising the steps of:
providing at least two samples each comprising distinct cell types;
performing single cell mass spectrometry imaging on at least two single cells in each sample in a predefined m/z range, wherein for each cell a minimum of one, preferably three, single cell mass spectrometry imaging datasets are obtained;
building a database comprising for each cell line the single cell mass spectrometry imaging data within the predefined m/z range and the corresponding subcellular localization data; and
constructing the recognition model by applying a dimensionality reduction algorithm to the reference database,
preferably wherein the dimensionality reduction algorithm is selected from Principal component analysis (PCA), Linear discriminant analysis (LDA), Multi-dimensional scaling (MDS), Singular value decomposition (SVD), Locally linear embedding (LLE), Isometric mapping (ISOMAP), Laplacian Eigenmap (LE), Independent component analysis (ICA) or t-Distributed Stochastic Neighbor Embedding (t-SNE),
preferably wherein the at least two samples comprising distinct cell types are at least two distinct cell lines.
